(19) United States
(12) Patent Application Publication
(10) Pub. No.: US 2024/0225806 A1
(43) Pub. Date: Jul. 11, 2024

(54) METHOD AND SYSTEM FOR MEASURING A PATIENT'S MOUTH

(71) Applicant: NEXUS DENTAL SYSTEMS, SOUTH PLAINFIEL, NJ (US)

(72) Inventor: Stanley Eugene Jones, Knightdale, NC (US)

(73) Assignee: NEXUS DENTAL SYSTEMS, SOUTH PLAINFIELD, NJ (US)

(21) Appl. No.: 18/407,982

(22) Filed: Jan. 9, 2024

Related U.S. Application Data
(60) Provisional application No. 63/479,044, filed on Jan. 9, 2023.

(51) Int. Cl. A61C 19/05; A61C 9/00; A61F 5/56

(52) U.S. Cl. CPC A61C 19/05; A61C 9/0006; A61F 5/566

(57) ABSTRACT

A system determines the optimal vertical and horizontal position of a patient's mandible for properly fitting an oral appliance for the treatment of obstructive sleep apnea or other conditions. Existing systems are difficult to use and result in inaccurate measurements. The present invention includes a double-sided vertical gauge for determining vertical adjustment, a set of progressively-sized keys for determining horizontal adjustment, and an arch fork. The unique designs of the vertical gauge and the keys facilitate easier and more accurate measurements.

### CROSS-REFERENCE TO RELATED APPLICATION

This application claims the benefit of priority to U.S. Provisional Patent Application Ser. No. 63/479,044, filed Jan. 9, 2023, which is incorporated herein by reference in its entirety.

### BACKGROUND OF THE INVENTION

The present invention relates to a method and system for measuring features of a patient's mouth. More particularly, the present invention relates to a method and system for measuring a patient's maximum mandible protrusion, bite set, and bite registration, to properly fit an oral appliance for the treatment of obstructive sleep apnea or other conditions.

Sleep apnea is a sleep disorder in which a person's breathing repeatedly stops and starts. Sleep apnea may cause loud snoring, gasps for air, awakening with a dry mouth, and headaches, among other symptoms. Obstructive sleep apnea is the most common form that occurs when the throat muscles relax.

The present invention, trade named Optimal Air, is an improvement on the Airway Metrics Snore Screener system, as described in U.S. Patent Publication No. 2010/0300457 A1. Unlike the Airway Metrics Snore Screener system, the present invention is not intended to diagnose snoring or sleep apnea. The present invention is instead designed to determine the optimal vertical and horizontal position of the mandible for oral appliance therapy and facilitate the bite set and bite registration measurements for fabrication of an oral appliance for the treatment of obstructive sleep apnea. The present invention allows a technician to make baseline horizontal and vertical measurements that can be used to fabricate a custom oral appliance.

The present invention is also an improvement on a device known as a George Gauge® device. A George Gauge device ostensibly allows a clinician to capture the protrusive bite registration and vertical opening of a patient without relying on the patient to achieve proper positioning. However, the design of a George Gauge device has proven unreliable in capturing measurements for mandible protrusion, bite set, or bite registration.

There is a need for a system that can take reliable mandible protrusion, bite set, or bite registration measurements for use in the fabrication of an oral appliance for the treatment of obstructive sleep apnea or other conditions.

### BRIEF DESCRIPTION OF THE DRAWINGS

FIG. 1 shows a top-left perspective view of a vertical gauge of an exemplary embodiment of the present invention.

FIG. 2 shows a bottom-right perspective view of a vertical gauge of an exemplary embodiment of the present invention.

FIG. 3 shows an arch fork of an exemplary embodiment of the present invention.

FIG. 4 shows a bottom-right perspective view of a key of an exemplary embodiment of the present invention.

FIG. 5 shows a top-left perspective view of a key of an exemplary embodiment of the present invention.

FIG. 6 shows a bite fork of an exemplary embodiment of the present invention, comprising an arch fork and a key.

FIG. 7 shows a case of an exemplary embodiment of the present invention, storing a vertical gauge and a plurality of keys.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the present invention.

Broadly, an embodiment of the present invention provides a system for measuring a patient's mouth by measuring features such as the patient's maximum mandible protrusion, bite set, and bite registration. These measurements may be used in the fabrication of an oral appliance that is properly sized for the patient for the treatment of obstructive sleep apnea or other conditions.

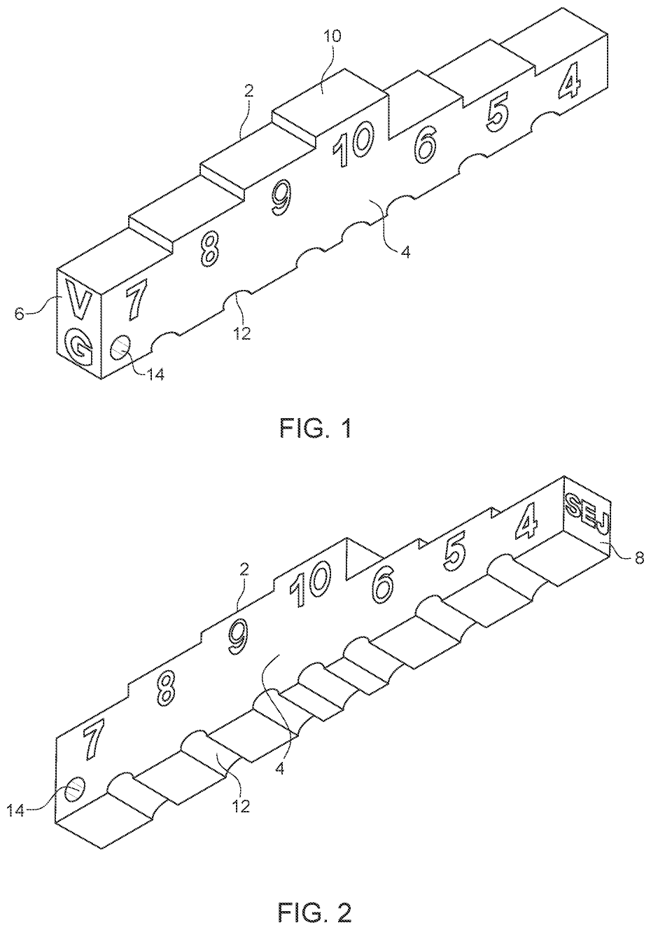

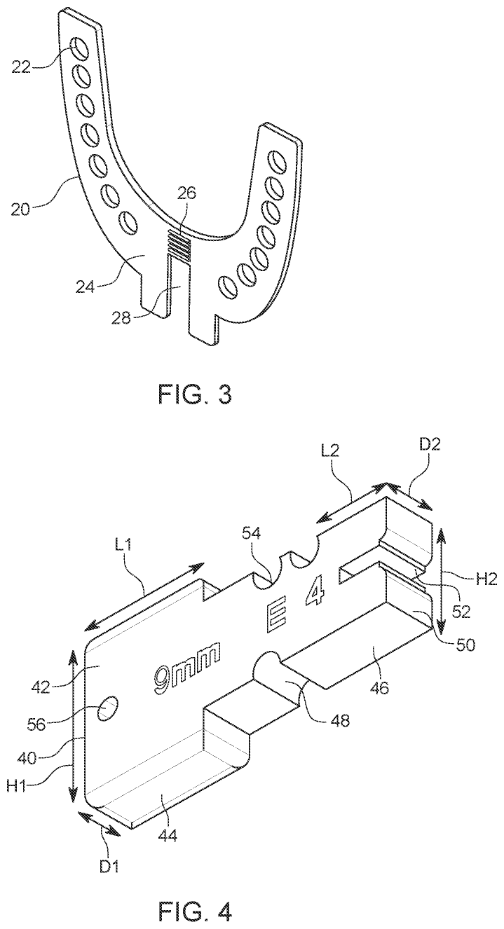

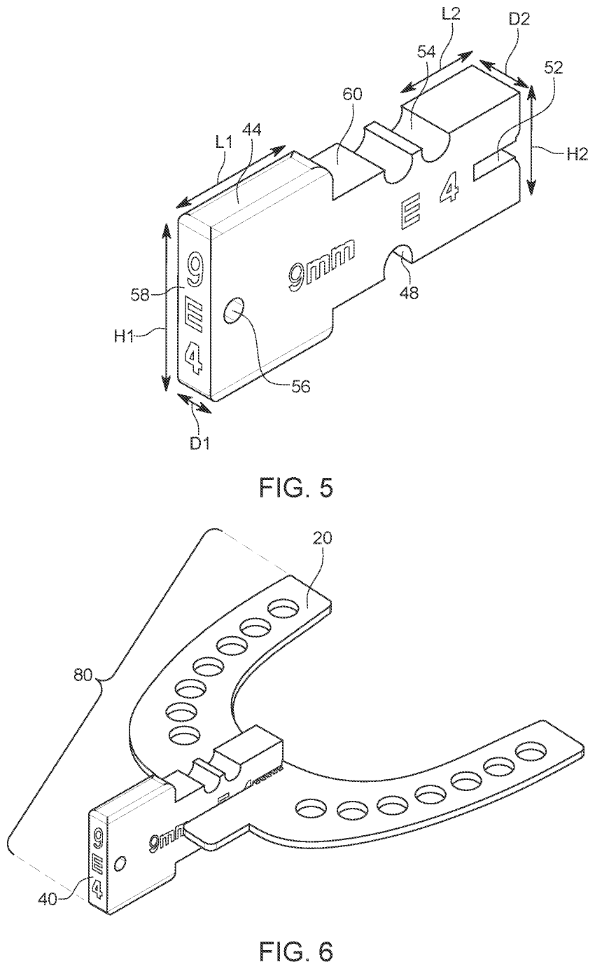

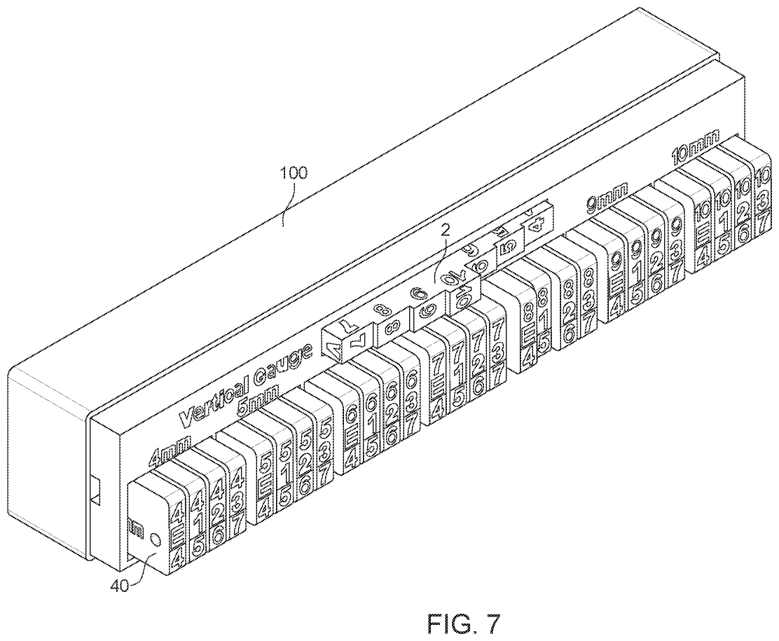

Referring now to FIGS. 1 through 7, the present invention may include the following components:
1. A vertical gauge, an example of which is depicted in FIGS. 1-2;
2. An arch fork, an example of which is depicted in FIG. 3;
3. A set of progressively sized keys that may be used for horizontal adjustment, an example of which is depicted in FIGS. 4-5;
4. A bite fork, comprising an arch fork and a key, an example of which is depicted in FIG. 6;
5. A case containing a vertical gauge and a set of progressively sized keys, an example of which is depicted in FIG. 7; and
6. A safety wire or dental floss (not depicted).

The present invention includes several unique design features.

In an exemplary embodiment, the vertical gauge 2 depicted in FIGS. 1-2 has a length of approximately 50 mm, a height of approximately 10 mm, and a width of approximately 5 mm. The top side of the vertical gauge has a series of steps 10. The vertical gauge has a left side 6 and a right side 8. The left side 6 of the vertical gauge 2 is labeled “VG,” indicating “Vertical Gauge.”

As shown in FIG. 1, starting at the left side 6 of the vertical gauge 2, the top side has four steps 10 starting at 7 mm in height and incrementing in height by 1 mm each step, labeled “7,” “8,” “9,” and “10” on the front surface 4 of the vertical gauge 2, followed by three steps 10 starting at 6 mm in height and decrementing by 1 mm each step, labeled “6,” “5,” and “4” on the front surface 4 of the vertical gauge 2. As the vertical gauge 2 has steps 10 descending along its length in both directions away from its center (i.e., starting from the center portion of the vertical gauge 2, steps 10 descend towards both the left side 6 and the right side 8), the vertical gauge 2 is double-sided. This offers an advantage over single-sided vertical gauges, where if a patient is on the highest level step of the vertical gauge, the entire vertical gauge up to that point must be placed into the patient's mouth, potentially causing discomfort and inaccurate measurements. With the double-sided feature of vertical gauge 2, only up to half of the vertical gauge 2 must ever be placed into the patient's mouth. Other variations of steps 10 are contemplated. For example, the vertical gauge may have three steps starting at 8 mm in height and incrementing by 1 mm each step, labeled 8, 9, and 10, followed by four steps starting at 7 mm in height and decrementing by 1 mm each step, labeled 7, 6, 5, and 4. The bottom side 16 of the vertical gauge 2 has semi-circular notches 12 extending along its length and positioned in the middle of the highest step 10 and at the intersection of each of the other steps 10. A patient may bite into these semi-circular notches 12, as explained in more detail below.

The vertical gauge 2 may be made of plastic. The shorter size is easier to fit in a patient's mouth and easy for the clinician to hold. The vertical gauge 2 also includes a hole 14 near one of its corners that allows the vertical gauge 2 or a set of keys 40 to be joined with wire or dental floss, providing a safeguard against dropping these components into a patient's mouth.

An arch fork 20 of the exemplary embodiment of the present invention is shown in FIG. 3. The arch fork 20 is designed to be inserted into a patient's mouth. The arch fork 20 has a thin profile with mirroring left and right sides 24, connected by a narrow center portion 26. Each side of the arch fork 20 has one or more holes 22 that may be used to allow flow of registration material. The arch fork 20 may be made of plastic. The center of the arch fork 20 forms a rectangular notch 28. The rectangular notch 52 of a key 40, described below, is configured to slide through the rectangular notch 28 of the arch fork 20 and onto its narrow center portion 26. The narrow center portion 26 ideally has ridged surfaces on each side to help keep a key 40 securely attached to the arch fork 20. When a key 40 and an arch fork 20 are attached, as shown in FIG. 6, these components are referred to collectively as a bite fork 80.

The exemplary embodiment of the present invention also includes a set of differently-sized keys. An individual key 40 is depicted in FIGS. 4-5. The key 40 has a base end 58, a tip end 50, a front side 42, and a rear side (not depicted), a top side, and a bottom side. The rear side is a mirror image of the front side 42. The key has a length of approximately 30 mm and a width of 5 mm. The base end 58 has a rectangular profile with a length L1, a height H1, and a width W1. Ideally, the length L1 is 12 mm, the height H1 is 14 mm, and the width is 5 mm. The base end 58 has top and bottom surfaces 44, which ideally have rounded edges to make handling the key 40 easier. The tip end 50 also has a rectangular profile with a length L2, a height H2, and a width W2. Ideally, the length L2 is 18 mm, the height H2 is 7 mm, and the width W2 is 5 mm. The tip end 50 has a top side 60 with at least two semi-circular notches 54 extending along its length. The tip end 50 also has a bottom side 46 with one or more semi-circular notches 48 extending along its length. A patient may bite into these notches 48 and 54, as explained in more detail below. The tip end 50 also includes a rectangular notch 52 extending through its center. This rectangular notch 52 allows a key 40 to attach to an arch fork 20.

The key 40 is labeled on both the front side 42 and the rear side. The labels and sizing of a key 40 are explained in more detail below. Each key 40 of the set of keys may increment in size by 1 mm.

The key 40 may be made of plastic. The unique shape of the key 40, including that the height H1 of the base end 58 is larger than the height H2 of the tip end 50 and that the top and bottom surfaces 44 has rounded edges, is designed to make it easier for a clinician to hold and use. The key 40 also includes a hole 56 on one of its ends that allows the key 40 and other keys or the vertical gauge 2 to be joined with wire or dental floss, providing a safeguard against dropping these components into a patient's mouth.

A case 100 is depicted in FIG. 7. The case 100 arranges the set of keys 40 in the order that they are to be used. The key sizes are clearly visible on the base end 58 of the keys. The case is also configured to store the vertical gauge 2. The case 100 is ideally made with a thick plastic material. A high thickness is desirable so that the case 100 does not break if it is dropped.

The components of the present invention may be manufactured using high-resolution 3D printing. This process achieves approximately 60 nm precision, but is expensive. The components of the present invention may alternatively be manufactured using injection molding. Injection molding is generally cheaper, but has quality control issues. A cheaper injection molding process may achieve approximately 200 nm precision, but a higher level of precision may be achieved with a more elaborate injection molding process. Achieving a high level of precision is important with these components because changes of even 10 nm may be noticeable to a patient.

Alternative to millimeters, components of the present invention may be dimensioned and labeled in microns.

In the exemplary embodiment, a method for use that simplifies the process of gauging the optimal vertical and horizontal positions of a patient's mandible for oral appliance therapy is as follows:

### Preparation—Items Needed

1. Optimal Air system
2. Periodontal probe
3. Nose cone or nasal dilator
4. Dental floss

### Measuring the Patient's Maximum Mandible Protrusion

1. With a periodontal probe, measure the patient's maximum protrusion of the mandible. Have the patient bring the patient's central incisors from edge to edge. Then ask the patient to protrude or extend the mandible as far forward as the patient can. With a periodontal probe, measure the distance from the facial of teeth #8 and #9, to the lingual of teeth #24 and #25. Record the number where you can refer to it as you set the patient's bite set with the Optimal Air system.
2. If the patient's maximum protrusion is 4 mm or less, vertical set measurement with the Optimal Air is critical. If the patient's maximum protrusion is 5 mm or greater, the vertical set measurement is not as critical of a measurement because the patient has enough horizontal range to set the patient's bite set.

### Bite Set Procedure

1. Explain to patient the procedure and why you are performing the procedure.
2. Have the patient sit up as straight as the patient can to 90 degrees.
3. Ask the patient to try and make a snore noise by slightly opening the patient's mouth.
4. Identify if the snore noise is nasal (upper airway) or throat (lower airway).
5. This can be accomplished by clamping or pinching the patient's nose and asking them to make a snore noise.
6. If the patient cannot make a snore noise, listen for air flow and air turbulence in the lower airway. Observe the patient to see how hard it is for the patient to try to make a snore noise. It will become harder for the patient to make a snore noise as progressing through the measurements.
7. Take the vertical gauge 2 from the Optimal Air system case. Explain to the patient that you will be having the patient bite on the various steps 10 of the vertical gauge 2 and try to make a snore noise.
8. Lay the patient back in the chair flat. Make sure that the patient's head is level to the floor. The patient needs to be laying as flat as possible, just like the patient would in a bed.
9. Determine which size nose cone or nasal dilator the patient will need. Slowly and gently place the cone or dilator in the patient's nasal openings. When you reach resistance, stop, and ask the patient to finish inserting the cone or dilator as far as the patient comfortable can.
10. Start with the 4 mm step 10 first. Have the patient open the patient's mouth and place the 4 mm step 10 on the vertical gauge 2 between teeth #8 and #9. Make sure that the edge of step 10 rests on the facial of these teeth. Then have the patient slowly bite down as you guide teeth #24 and #25 in the semi-circular notch 12 at the bottom of the 4 mm step 10 on the vertical gauge 2.
11. Ask the patient to make a snore noise. Listen for a reduction of the snore noise and a greater airflow. If the patient cannot make a snore noise, listen for air turbulence reduction, and observe if it is harder for them to attempt to make the snore noise.
12. Have the patient open the patient's mouth and remove the vertical gauge 2. After taking a measurement, ask the patient to swallow and relax. Ask how the patient feels and if the patient is experiencing any pain.
13. Repeat steps 10 through 12 for the other steps 10, 5 mm through 9 mm. Note any changes in the sound of the snore noise, air turbulence, and how hard it is for the patient to attempt to make the snore noise.
14. When you have reduced the snore noise or air turbulence the best you can, stop at that step and record the measurement as your vertical set. Example: vertical set=6 mm.
15. Now that you have the vertical set measurement, go to the Optimal Air system case 100 and choose the first horizontal key 40 that corresponds to the vertical measurement. Start with “E”. “E” stands for “edge to edge.” Example: vertical set measurement=6 mm. Go to the 6 mm horizontal keys 40 and choose the 6/E key 40.
16. Have the patient open the patient's mouth and guide the “E” notch 54 on or between teeth #8 and #9. Have the patient bite down slowly as the patient protrudes the patient's mandible forward. Guide teeth #24 and #25 or between them into the notch 48 on the bottom of the horizontal key.
17. Have the patient make a snore noise and listen for the sound level of the snore or air turbulence. Note if this was better or worse.
18. Have the patient open and remove the horizontal key 40. Choose the 6 mm 1 horizontal key 40 (Example: 6/1). Repeat steps 16 and 17, but this time have the patient bite into the upper notch 54 labeled “1” with teeth #24 or #25 or between them. Again, listen to see if the snore noise or air turbulence is better or the same.

Continue to the next notch 54 until you get the lowest snore noise or turbulence you can. Once you have the eliminated or reduced to lowest the snore sound and air turbulence, it is time to take a bite registration. Example: record your bite set as follows: vertical set: 6 mm, horizontal set: 2 mm.

### Bite Registration Procedure

1. Scanning the Bite: Scan the lower arch and the upper arch. Then have the patient bite on the horizontal key 40 that was the best measurement, and scan in an oval pattern on both sides from the 2nd premolars to the 1st molar. The bite will align in the vertical and horizontal set you have chosen.
2. Bite Registration Material Bite: Place an arch fork 20 in the front slot of the horizontal key 40 you have chosen for the patient's bite set, forming a bite fork 80. Apply bite registration material to both sides of the bite-fork 80. Have the patient bite into the chosen notches 48 and 54 on the horizontal key 40. Fill in the spaces between the teeth and the bite-fork 80 on the mandibular and maxillary sides of the bite-fork, and then have the patient close the patient's lips together. Let the material set and remove.

The following table further describes the differences between the present invention and the Airway Metrics Snore Screener system.

| Airway Metrics Snore Screener | Optimal Air |
|---|---|
| (US 2010/0300457 A1) | (the present invention) |
| Although the Airway Metrics system is | Optimal Air is a system for determining |
| defined in the publication as a “method | the optimal vertical and horizonal position |
| and apparatus for treating sleep apnea,” | of the mandible for the beginning of oral |
| that is not how the system is used in | appliance therapy. The system allows a |
| dental labs. The Airway Metrics system | technician to make baseline horizonal and |
| cannot treat sleep apnea at all. | vertical measurements for the fabrication |
| | of a custom oral appliance. |
| The Airway Metrics system uses several | The Optimal Air keys are printed on both |
| “airway dilation simulators blocks” for | sides and are not reversible, so the |
| gauging protrusion of the mandible, with | number on the gauge is the measure to |
| notches in two or three positions each. | be recorded, eliminating the possibility of |
| They are described as reversible, but in | error. |
| practice they only have numbers on one | |
| side, forcing a dentist to manually | |
| compute the position if used in the | |
| reverse. | |
| The Airway Metrics system uses simulator | The Optimal Air horizontal keys are larger |
| blocks (horizontal set) that are small and | and have been designed with a “key” style |
| hard to manage, resulting in frequent | head, which makes them easier for a |
| dropping during use. | clinician to hold and use. |
| The Airway Metrics system (comparable | The Optimal Air has a unique double- |
| to the Optimal Air vertical gauge) has four | sided vertical gauge with seven steps of |
| steps of vertical adjustment: 2 mm, 4 mm, | vertical adjustment (4 mm, 5 mm, 6 mm, |
| 8 mm, and 12 mm. | 7 mm, 8 mm, 9 mm, and 10 mm). This |
| | allows more precise fitting, for patient |
| | comfort and clinical effect. |
| The Airway Metrics system (comparable | The Optimal Air vertical gauge is shorter |
| to the Optimal Air vertical gauge) is too | in length and height and is easier to use |
| long and the gauge goes up to 12 mm, | by the clinician when setting the patient's |
| which is too high, making it cumbersome | bite for the patient's custom oral sleep |
| for the clinician to use. | appliance. |
| The metric numbers on all components of | The Optimal Air system displays metric |
| the Airway Metrics system are very small | numbers large on both sides and the top |
| and only visible on the left side of the | of all the components in the system, |
| components, making them hard to see | making them easier to use by the |
| and use by the clinician | clinicians. |
| The Airway Metrics system is packaged in | Optimal Air is packaged and laid out in the |
| a confusing order that makes it difficult for | systematic order of process of setting the |
| the clinician to find the proper component | bite. This makes the system easy to learn |
| when setting the patient's bite. | and use. |
| The Airway Metrics system components | Optimal Air has large round notches that |
| have small bite grooves for the patient's | allow for better alignment and can |
| maxillary and mandibular incisors to fit in. | compensate for the unevenness of the |
| To work effectively the patient's central | patient's central incisors. This helps the |
| incisors must be straight and level, which | patient to have a more comfortable bite |
| can cause the patient's bite to shift | set in the Optimal Air components and |
| laterally during the bite registration | assist the clinician with proper alignment |
| process. A custom oral sleep appliance | of the bite set. |
| created from these measurements can be | |
| shifted from the patient's proper bite, | |
| putting excessive stress on the | |
| temporomandibular joint and causing the | |
| patient's bit to be misaligned. | |
| The Airway Metrics system can be off by | Optimal Air is accurate within 50 microns |
| up to 300 microns due to mass | |
| production. | |
| The Airway Metrics system places the | Optimal Air is designed so the horizontal |
| simulator blocks on the central incisors of | key is placed on the maxillary central |
| the mandible, bringing it up to meet the | incisors, with the mandible coming up to |
| maxilla. This can result in movement of | meet it during measurement. Because the |
| the simulator blocks, resulting in imprecise | maxilla does not move, this produces a |
| measurement. | much more accurate and reliable |
| | measurement. |
| The Airway Metrics system simulator | Optimal Air has a straightforward |
| blocks are reversible, allowing more | progression system, with no flipping or |
| variations, but the labeling is not clear. | manipulation required for a precise |
| | measurement (one key for each |
| | measurement). This results in fewer |
| | mistakes, easy for dental assistant to |
| | learn |
| The Airway Metrics system has no built-in | Optimal Air has a built-in safety system |
| safety system, thus making it possible that | that avoids the patient from choking or |
| a patient could swallow or choke on one | swallowing one of the components during |
| of the components, posing a health | use. A small hole located in the lower right |
| hazard. | corner of each component of the Optimal |
| | Air system allows a 12-to-16-inch piece of |
| | dental floss to be threaded through. In |
| | case the patient shifts the patient's bite, or |
| | the clinician drops the component, the |
| | dental floss can be used to retract the |
| | component quickly. |
| The vertical gauge of the Airway Metrics | The vertical gauge in Optimal Air has |
| system lacks notches along its bottom | notches along the bottom of its vertical |
| surface. With nothing to bite into on the | gauge, allowing a patient to bite into both |
| bottom surface, a patient's mandibular | the top and lower surfaces of the vertical |
| incisors may slip along the bottom | gauge. This helps ensure accuracy. Even |
| surface, leading to inaccurate | slight improvements in vertical adjustment |
| measurements. There is no way to know | means that there is less variation needed |
| whether differences in a patient's snoring | in mandibular adjustment. |
| sound at different steps on the vertical | |
| gauge is due to vertical adjustment or | |
| mandibular adjustment. | |

It should be understood, of course, that the foregoing relates to exemplary embodiments of the invention and that modifications may be made without departing from the spirit and scope of the present invention.

What is claimed is:

1. A system for measuring a patient's mouth, comprising:
a vertical gauge; and
one or more keys configured to be attached to an arch fork,
wherein each key comprises a base end and a tip end, and
wherein the base end has a vertical height H1 and the tip end has a vertical height H2, and the vertical height H1 is greater than the vertical height H2.

2. The system of claim 1, wherein the vertical gauge comprises a left side, a right side, a center portion between the left side and the right side, a top side, and a bottom side, the top side comprising two or more steps.

3. The system of claim 2, wherein the vertical gauge is double-sided, with one or more of the steps descending along the length of the vertical gauge from the center portion to the left side and one or more of the steps descending along the length of the vertical gauge from the center portion to the right side.

4. The system of claim 3, wherein the vertical gauge comprises seven steps in 1 mm increments.

5. The system of claim 3, wherein the vertical gauge comprises two or more notches on the bottom side corresponding to each step, wherein each of the two or more notches on the bottom side of the vertical gauge is configured to receive a patient's mandibular incisors.

6. The system of claim 1, wherein the base end of each key has rounded edges.

7. The system of claim 1, wherein each key has a hole near the base end.

8. The system of claim 1, wherein each key has a front side and a rear side connecting the base end and the tip end, and measurement information is printed on both the front side and the rear side.

9. The system of claim 1, wherein each key has a top side and a bottom side, the top side comprising two or more notches for receiving a patient's maxillary incisors, and the bottom side comprising a single notch for receiving a patient's mandibular incisors.

10. The system of claim 9,
wherein the vertical gauge comprises a left side, a right side, a center portion between the left side and the right side, a top side, and a bottom side, the top side comprising two or more steps;
wherein the vertical gauge is double-sided, with one or more of the steps descending along the length of the vertical gauge from the center portion to the left side and one or more of the steps descending along the length of the vertical gauge from the center portion to the right side; and
wherein the vertical gauge comprises two or more notches on the bottom side corresponding to each step, wherein each of the two or more notches on the bottom side of the vertical gauge is configured to receive a patient's mandibular incisors.

11. A method for measuring a patient's mouth, comprising:
determining an optimal vertical position of a patient's mandible using a vertical gauge; and
determining an optimal horizontal position of the patient's mandible using one or more keys configured to be attached to an arch fork,
wherein each of the one or more keys corresponds to the optimal vertical position of the patient's mandible,
wherein each key comprises a base end and a tip end, and
wherein the base end has a vertical height H1 and the tip end has a vertical height H1, and the vertical height H1 is greater than the vertical height H2.

12. The method of claim 11, wherein the vertical gauge comprises a left side, a right side, a center portion between the left side and the right side, a top side, and a bottom side, the top side comprising two or more steps.

13. The method of claim 12, wherein the vertical gauge is double-sided, with one or more of the steps descending along the length of the vertical gauge from the center portion to the left side and one or more of the steps descending along the length of the vertical gauge from the center portion to the right side.

14. The method of claim 12, wherein the vertical gauge comprises seven steps in 1 mm increments.

15. The method of claim 13,
wherein the vertical gauge comprises two or more notches on the bottom side corresponding to each step;
wherein each of the two or more notches on the bottom side of the vertical gauge is configured to receive a patient's mandibular incisors; and
wherein determining the optimal vertical position of the patient's mandible using the vertical gauge comprises:
(i) having the patient place the patient's maxillary incisors onto a step on the top side of the vertical gauge;
(ii) having the patient bite using the patient's mandibular incisors onto a corresponding notch on the bottom side of the vertical gauge;
(iii) measuring at least one of a patient's snoring noise and airflow; and
(iv) repeating (i)-(iii) for different steps on the top side of the vertical gauge to determine the optimal vertical position of the patient's mandible where the snoring noise is minimized or airflow is maximized.

16. The method of claim 11, wherein the base end of each key has rounded edges.

17. The method of claim 11, wherein each key has a hole near the base end.

18. The method of claim 11, wherein each key has a front side and a rear side connecting the base end and the tip end, and measurement information is printed on both the front side and the rear side.

19. The method of claim 11,
wherein each key has a top side and a bottom side, the top side comprising two or more notches for receiving a patient's maxillary incisors, and the bottom side comprising a single notch for receiving a patient's mandibular incisors; and
wherein determining the optimal horizontal position of the patient's mandible using one or more keys comprises:
(i) having the patient place the patient's maxillary incisors onto a first notch of the two or more notches on the top side of a first key;
(ii) having the patient place bite using the patient's mandibular incisors onto the notch on the bottom side of the first key;
(iii) measuring a patient's snoring noise or airflow; and
(iv) repeating (i)-(iii) for different notches on the top side of the first key;
(v) repeating (i)-(iv) for each of the one or more keys to determine the optimal horizontal position of the patient's mandible where snoring noise is minimized or airflow is maximized.

20. The method of claim 19,
wherein the vertical gauge comprises two or more notches on the bottom side corresponding to each step;
wherein each of the two or more notches on the bottom side of the vertical gauge is configured to receive a patient's mandibular incisors; and
wherein determining the optimal vertical position of the patient's mandible using the vertical gauge comprises:
(i) having the patient place the patient's maxillary incisors onto a step on the top side of the vertical gauge;
(ii) having the patient bite using the patient's mandibular incisors onto a corresponding notch on the bottom side of the vertical gauge;
(iii) measuring at least one of a patient's snoring noise and airflow; and
(iv) repeating (i)-(iii) for different steps on the top side of the vertical gauge to determine the optimal vertical position of the patient's mandible where the snoring noise is minimized or airflow is maximized.
